(19) United States
(12) Patent Application Publication
(10) Pub. No.: US 2026/0124272 A1
(43) Pub. Date: May 7, 2026

(54) METHODS FOR INCREASING TYPE I, III, AND V COLLAGEN IN AN ANIMAL

(71) Applicant: Société des Produits Nestlé S.A., vevey (CH)

(72) Inventors: Yuanlong Pan, Chesterfield, MO (US); Rondo P. MIDDLETON, CREVE COEUR, MO (US); Chang Woon Jang, Chesterbrook, PA (US)

(73) Assignee: Société des Produits Nestlé S.A., vevey (CH)

(21) Appl. No.: 19/311,247

(22) Filed: Aug. 27, 2025

Related U.S. Application Data
(60) Provisional application No. 63/716,315, filed on Nov. 5, 2024.

(51) Int. Cl. A61K 38/16; A23K 20/147; A23K 20/158; A23K 20/163; A23K 50/42; A61K 31/718; A61K 38/17

(52) U.S. Cl. CPC A61K 38/168; A23K 20/147; A23K 20/158; A23K 20/163; A23K 50/42; A61K 31/718; A61K 38/1703; A61K 38/1706; A61K 38/1709

(57) ABSTRACT

The present disclosure provides compositions and methods for increasing type I, III, and V collagen in an animal can comprise administering a food composition to the animal, wherein the food composition comprises from about 30% to about 65% protein, from about 0.1% to about 30% starch, and from about 10% to about 40% fat. Additionally, the food composition can have a protein to starch ratio of at least 2:1.

### CROSS REFERENCE TO RELATED APPLICATION

This application claims priority to U.S. Provisional Application Ser. No. 63/716,315 filed Nov. 5, 2024 the disclosure of which is incorporated in its entirety herein by this reference.

### BACKGROUND

The skin is the major external protective organ of animals. For pets, a healthy coat is generally considered to be indicative of a healthy pet; and vice versa. For this reason, coat problems are one of the primary causes for pet owners to take their pets to veterinarians. These problems need not necessarily manifest themselves only in the coat but may appear in the form of dermatosis such as skin flaking, itchiness, skin lesions, and general dryness of the skin. These are often indicative of a malaise of the protective system provided by the skin and coat as an integrated functioning unit.

Much can be done to maintain or improve the pet's condition by feeding it a complete and balanced food. This provides the essential nutrients and minerals needed to maintain its skin and coat. However, in view of the many agents in the environment that may damage a pet's skin and coat system, feeding the pet a complete and balanced food may not always be sufficient. Therefore, many supplements are commercially available for feeding to pets for improving their skin and coat. These supplements are often of questionable efficacy.

For example, zinc and linoleic acid are believed to improve the skin of pets. However, the levels of zinc required to provide a benefit can be in excess of those permitted in pet foods in certain markets. Also, high levels of linoleic acid are believed by some veterinarians to be pro-inflammatory.

Additionally, eye health is a concern for animals, especially aging animals. Supplements are commonly used to address eye health, but they can be costly and problematic to administer.

As such, there remains a need for a method of improving or maintaining eye health and skin health of animals.

### SUMMARY

In one embodiment, a method for increasing type I, III, and V collagen in an animal can comprise administering a food composition to the animal, wherein the food composition comprises from about 30% to about 65% protein, from about 0.1% to about 30% starch, and from about 10% to about 40% fat. Additionally, the food composition can have a protein to starch ratio of at least 2:1.

Other and further objects, features, and advantages of the invention will be readily apparent to those skilled in the art.

### DETAILED DESCRIPTION

### Definitions

The term “animal” means any animal that has a need for the health benefits described herein, including human, avian, bovine, canine, equine, feline, hircine, lupine, murine, ovine, or porcine animals. In one aspect, the animal can be a mammal.

The term “companion animal” means domesticated animals such as cats, dogs, rabbits, guinea pigs, ferrets, hamsters, mice, gerbils, horses, cows, goats, sheep, donkeys, pigs, and the like. In one aspect, the companion animal can be a canine. In another aspect, the companion animal can be a feline.

The term “aging” means being of advanced age such that the animal has exceeded 50% of the average lifespan for its particular species and/or breed within a species. For example, if the average lifespan for a given breed of dog is 10 years, then a dog within that breed greater than 5 years old would be considered “aging” for purposes herein. Additionally, for example, if the average lifespan for a breed of cat is 15 years, then a cat within that breed greater than 7.5 years old would be considered “aging” for the purposes herein. In an embodiment, the compositions and methods disclosed herein involve a senior animal, e.g. a senior dog or a senior cat. Animals, such as dogs and cats, are considered senior in the last 25% of their lives. As noted herein, the life span of a dog or a cat depends on its size and/or its breed, and a senior dog or senior cat can be determined based on the above calculation but using the numerical value of 75% such that the age threshold is exceeding 75% of the average lifespan.

The terms “treating”, “treat”, and “treatment” embrace both preventative, i.e., prophylactic, and palliative treatment.

The term “health and/or wellness of an animal” means the complete physical, mental, and social well-being of the animal, not merely the absence of disease or infirmity.

The term “in conjunction” means that the food composition, components thereof, or other compositions disclosed herein are administered to an animal (1) together in a single food composition or (2) separately at the same or different frequency using the same or different administration routes at about the same time or periodically. “Periodically” means that the food composition, components thereof, or other compositions are administered on a schedule acceptable for specific compounds or compositions. “About the same time” generally means that the food composition, components thereof, or other compositions are administered at the same time or within about 72 hours of each other.

The term “food” or “food product” or “food composition” means a product or composition that is intended for ingestion by an animal, including a human, and provides nutrition to the animal.

The term “non-ketogenic food composition” refers to a food composition that does not induce ketosis in the animal where ketosis in achieved by metabolizing fat into ketone bodies such as high fat, low carbohydrate compositions. Additionally, in aspect, the non-ketogenic food composition can exclude components that induce ketosis by oral ingestion of ketone precursors such as medium chain triglycerides.

The term “carbohydrate” refers to carbohydrates that are digestible, e.g. sugars and starches, and does not include fiber, e.g. cellulose or fermentable fibers.

The term “starch” refers to a specific type of carbohydrate, i.e., a complex carbohydrate, or polysaccharide, made up of multiple monosaccharides.

The term “regular basis” means at least monthly administration and, in one aspect, at least weekly administration. More frequent administration or consumption, such as twice or three times weekly, can be performed in certain embodiments. In one aspect, an administration regimen can comprise at least once daily consumption.

The term “long-term administration” means periods of repeated administration or consumption in excess of one month. Periods of longer than two, three, or four can be used for certain embodiments. Also, more extended periods that include longer than 5, 6, 7, 8, 9, or 10 months can be used. Periods in excess of 11 months or 1 year can also be used. Longer term use extending over 1, 2, 3, or more years can also be included in the invention. For certain aging animals, the animal will continue consuming on a regular basis for the remainder of its life. Sometimes this is referred to as consumption for “extended” periods.

The term “single package” means that the components of a kit are physically associated in or with one or more containers and considered a unit for manufacture, distribution, sale, or use. Containers include, but are not limited to, bags, boxes, cartons, bottles, packages such as shrink wrap packages, stapled or otherwise affixed components, or combinations thereof. A single package may be containers of food compositions, or components thereof, physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The term “about” means plus or minus 20% of a numeric value; in one aspect, plus or minus 10%; in another aspect, plus or minus 5%; and in one specific aspect, plus or minus 2%. For example, in one aspect where about is plus or minus 20% of a numeric value, the phrase “from about 10% to about 20%” could include a range from 8% to 24% or 12% to 16%, include any subranges therein.

As used herein, embodiments, aspects, and examples using “comprising” language or other open-ended language can be substituted with “consisting essentially of” and “consisting of” embodiments.

The term “complete and balanced” when referring to a food composition means a food composition that contains all known required nutrients in appropriate amounts and proportions based on recommendations of recognized authorities in the field of animal nutrition, and are therefore capable of serving as a sole source of dietary intake to maintain life or promote production, without the addition of supplemental nutritional sources. Nutritionally balanced pet food and animal food compositions are widely known and widely used in the art, e.g., complete and balanced food compositions formulated according to standards established by the Association of American Feed Control Officials (AAFCO).

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a supplement”, “a method”, or “a food” includes a plurality of such “supplements”, “methods”, or “foods.” Similarly, the words “comprise”, “comprises”, and “comprising” are to be interpreted inclusively rather than exclusively. Likewise, the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, certain compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### DETAILED DESCRIPTION

The present methods and compositions are based upon the discovery that specific food compositions have been found to enhance type I, III, and V collagen in an animal. Specifically, the present food compositions utilize a specific composition with a ratio of protein to starch that enhances type I, III, and V collagen to provide health benefits as compared to known dietary regimens such as moderate protein and carbohydrate food compositions, or the use of costly additives or supplements. However, the use of such additives and supplements can be used in conjunction with the methods and compositions disclosed herein. Such health benefits include improved skin, improved coat, improved hair, supporting skin health and function, maintaining skin health and function, treating signs of skin aging, reducing or delaying signs of skin aging, treating dermatosis, decrease of itchy skin, improving eye health, supporting or maintaining eye health, improving cornea health, supporting or maintaining cornea health, treating or preventing cornea disease, and combinations thereof. As used herein, “signs of skin aging” include, but is not limited to, wrinkles, age spots, dryness, bruising, skin tags, dull skin, dull coat, thin skin, loss of volume, graying/whitening hair, loss of hair, thin hair, callus formation, skin odor, brittle nails, thickened footpads, lumps and bumps, lipomas, rough skin, sagging skin, loss of skin elasticity, loss of skin tone, skin discoloration, dark circles, hyperpigmentation, and slower healing.

In accordance with these discoveries, in one embodiment, a method for increasing type I, III, and V collagen in an animal can comprise administering a food composition to the animal, wherein the food composition comprises: from about 30% to about 65% protein, from about 0.1% to about 30% starch, and from about 10% to about 40% fat. Generally, the food composition can have a protein to starch ratio of at least 2:1.

Generally, such ratios of protein to starch can range from about 2:1 to about 10:1, although ratios higher than 10:1 could be considered. In one embodiment, the ratio of protein to starch ranges from about 2:1 to about 6:1. In one aspect, the ratio of protein to starch can range from about 2.5:1 to about 6:1. In other aspects, the ratio of protein to starch can range from about 2.5:1 to about 4:1, from about 2:1 to about 3:1, from about 2.5: to about 3.5:1, about 3:1 to about 4:1, or about 3:1 to about 5.1. Other subranges can also be used. Further, ratios of protein to carbohydrates can also be independently present in the above ratios. For example, a composition can have a protein to carbohydrate ratio of about 2:1 to 3:1 and a protein to starch ratio of about 2.5:1 to about 3.5:1.

Generally, the present compositions comprise a protein. The protein can be crude protein material and may comprise plant proteins such as soybean meal, soy protein concentrate, corn gluten meal, wheat gluten, cottonseed, and peanut meal, or animal proteins such as casein, albumin, and meat protein. Examples of meat protein useful herein include beef, pork, lamb, poultry, fish, and mixtures thereof. In one embodiment, the food compositions can comprise the protein in amounts from about 30%, 35%, 40%, 45%, 50%, 55%, or even 60% to about 35%, 40%, 45%, 50%, 55%, 60%, or even 65%, including various subranges within these amounts. In one aspect, the protein can be from about 40% to about 55% of the food composition.

Generally, any type of carbohydrate, including those as a source of starch, can be used in the food compositions. Examples of suitable carbohydrates include grains or cereals such as rice, corn, millet, sorghum, alfalfa, barley, soybeans, canola, oats, wheat, rye, triticale and mixtures thereof. The compositions may also optionally comprise other materials such as dried whey and other dairy by-products. In one embodiment, the starch can comprise from about 0.1% to about 30% of the food composition. In another embodiment, the carbohydrates can comprise from about 0.1% to about 30% of the food composition. In still other embodiments, the starch and/or carbohydrates comprise from about 10% to about 20% of the food compositions. In other aspects, the starch and/or carbohydrates can be present in amounts from about 5%, 6%, 7%, 8%, 9%, 10%, 11%, 12%, 13%, 14%, 15%, 16%, 17%, 18%, 19%, or even 20%, to about 6%, 7%, 8%, 9%, 10%, 11%, 12%, 13%, 14%, 15%, 16%, 17%, 18%, 19%, 20%, 21%, 22%, 23%, 24%, or 25%. Additionally, the starch or carbohydrates can be present in these amounts or greater.

Generally, the food compositions include fat. Examples of suitable fats include animal fats and vegetable fats. In one aspect, the fat source can be an animal fat source such as tallow or poultry fat. Vegetable oils such as corn oil, sunflower oil, safflower oil, grape seed oil, soybean oil, olive oil and other oils rich in monounsaturated and polyunsaturated fatty acids may also be used. In one embodiment, the food compositions can comprise the fat in amounts from about 10%, 15%, 20%, 25%, 30%, or even 35% to about 15%, 20%, 25%, 30%, 35%, or even 40%, including various subranges within these amounts. In one aspect, the fat comprises from about 10% to about 30% of the food composition.

The administration can be performed on an as-needed basis, an as-desired basis, a regular basis, or intermittent basis. In one aspect, the food composition can be administered to the animal on a regular basis. In one aspect, at least weekly administration can be performed. More frequent administration or consumption, such as twice or three times weekly, can be performed in certain embodiments. In one aspect, an administration regimen can comprise at least once daily consumption. In one embodiment, the administration can be long-term.

According to the presently described methods, administration, including administration as part of a dietary regimen, can span a period ranging from parturition through the adult life of the animal. In various embodiments, the animal can be a human or companion animal such as a dog or cat. In certain embodiments, the animal can be a young or growing animal. In other embodiments, administration can begin, for example, on a regular or extended regular basis, when the animal has reached more than about 10%, 20%, 30%, 40%, or 50% of its projected or anticipated lifespan. In some embodiments, the animal can have attained 40, 45, or 50% of its anticipated lifespan. In yet other embodiments, the animal can be older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan. In one aspect, the animal can be an aging animal. In another aspect, the animal can be a senior animal.

Such administration can be performed for a time required to accomplish one or more objectives described herein, e.g., improved skin, improved coat, supporting skin health and function, maintaining skin health and function, treating dermatosis, decrease of itchy skin, and combinations thereof. Other administration amounts may be appropriate and can be determined based on the animal's initial weight as well as other variables such as species, gender, breed, age, desired health benefit, etc.

The moisture content for such food compositions varies depending on the nature of the food composition. The food compositions may be dry compositions (e.g., kibble), semi-moist compositions, wet compositions, or any mixture thereof. In one embodiment, the composition can be a pet food composition, and in one aspect, can be a complete and nutritionally balanced pet food. In this embodiment, the pet food may be a “wet food”, “dry food”, or food of “intermediate moisture” content. “Wet food” describes pet food that is typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one embodiment, the compositions can have moisture content from about 5% to about 20%. Dry food products include a variety of foods of various moisture contents, such that they are relatively shelf-stable and resistant to microbial or fungal deterioration or contamination. Also, in one aspect, dry food compositions can be extruded food products for either humans or companion animals.

The food compositions may also comprise one or more fiber sources. Such fiber sources include fiber that is soluble, insoluble, fermentable, and nonfermentable. Such fibers can be from plant sources such as marine plants, but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefits described herein or to the immune system of an animal.

In some embodiments, the ash content of the food composition ranges from less than 1% to about 15%. In one aspect, the ash content can be from about 5% to about 10%.

Generally, the food composition can be suitable for consumption by an animal, including humans and companion animals such as dogs and cats, as a meal, component of a meal, a snack, or a treat. Such compositions can include complete foods intended to supply the necessary dietary requirements for an animal. Examples of such food compositions include but are not limited to dry foods, wet foods, drinks, bars, frozen prepared foods, shelf prepared foods, and refrigerated prepared foods.

Food compositions may further comprise one or more substances such as vitamins, minerals, antioxidants, probiotics, prebiotics, salts, and functional additives such as palatants, colorants, emulsifiers, and antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat-soluble vitamins as A, D, E, and K. Inulin, amino acids, enzymes, coenzymes, and the like may be useful to include in various embodiments.

The present methods for increasing type I, III, and V collagen in an animal can provide a health benefit to the animal. As such, the method can include identifying an animal in need of a health benefit. The health benefit can be a skin health benefit and/or a eye health benefit. As such, in one embodiment, the method can include identifying an animal in need of skin health and/or eye health, including preventing, treating, supporting, maintaining, improving, and/or delaying issues related thereto. In another embodiment, the health benefit can include improved skin, improved coat, improved hair, supporting skin health and function, maintaining skin health and function, treating signs of skin aging, reducing or delaying signs of skin aging, treating dermatosis, decrease of itchy skin, improving eye health, supporting or maintaining eye health, improving cornea health, supporting or maintaining cornea health, treating or preventing cornea disease, and combinations thereof.

In various embodiments, the food compositions contain at least one of (1) one or more probiotics; (2) one or more inactivated probiotics; (3) one or more components of inactivated probiotics that promote health benefits similar to or the same as the probiotics, e.g., proteins, lipids, glycoproteins, and the like; (4) one or more prebiotics; and (5) combinations thereof. The probiotics or their components can be integrated into the food compositions (e.g., uniformly or non-uniformly distributed in the compositions) or applied to the food compositions (e.g., topically applied with or without a carrier). Such methods are known to skilled artisans, e.g., U.S. Pat. No. 5,968,569 and related patents.

Typical probiotics include, but are not limited to, probiotic strains selected from Lactobacilli, Bifidobacteria, or Enterococci, e.g., Lactobacillus reuteri, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp. In some embodiments, the probiotic strain can be selected from the group consisting of Lactobacillus reuteri (NCC2581; CNCM I-2448), Lactobacillus reuteri (NCC2592; CNCM I-2450), Lactobacillus rhamnosus (NCC2583; CNCM I-2449), Lactobacillus reuteri (NCC2603; CNCM I-2451), Lactobacillus reuteri (NCC2613; CNCM I-2452), Lactobacillus acidophilus (NCC2628; CNCM I-2453), Bifidobacterium adolescentis (e.g., NCC2627), Bifidobacterium sp. NCC2657 or Enterococcus faecium SF68 (NCIMB 10415). Generally, the food compositions can contain probiotics in amounts sufficient to supply from about 104 to about 1012 cfu/animal/day, in one aspect, from 105 to about 1011 cfu/animal/day, and in one specific aspect, from 107 to 1010 cfu/animal/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390, EP1482811B1, EP1296565B1, and U.S. Pat. No. 6,929,793. In one embodiment, the probiotic can be Enterococcus faecium SF68 (NCIMB 10415). In another embodiment, the probiotics can be encapsulated in a carrier using methods and materials known to skilled artisans.

As stated, the food compositions may contain one or more prebiotics, e.g., fructo-oligosaccharides, gluco-oligosaccharides, galacto-oligosaccharides, isomalto-oligosaccharides, xylo-oligosaccharides, soybean oligosaccharides, lactosucrose, lactulose, and isomaltulose. In one embodiment, the prebiotic can be chicory root, chicory root extract, inulin, or combinations thereof. Generally, prebiotics can be administered in amounts sufficient to positively stimulate the healthy microflora in the gut and cause these “good” bacteria to reproduce. Typical amounts range from about one to about 10 grams per serving or from about 5% to about 40% of the recommended daily dietary fiber for an animal. The probiotics and prebiotics can be made part of the composition by any suitable means. Generally, the agents can be mixed with the composition or applied to the surface of the composition, e.g., by sprinkling or spraying. When the agents are part of a kit, the agents can be admixed with other materials or in their own package. Typically, the food composition contains from about 0.1 to about 10% prebiotic, in one aspect, from about 0.3 to about 7%, and in one specific aspect, from about 0.5 to 5%, on a dry matter basis. The prebiotics can be integrated into the compositions using methods known to skilled artisans, e.g., U.S. Pat. No. 5,952,033.

Postbiotics useful for the present invention include, but are not limited to, metabolites or components produced by microbiota that have an impact on human health or inactivated probiotics, including any inactivated form of the probiotics listed herein, as well as short-chain fatty acids, microbial cell fragments, extracellular polysaccharides, cell lysates, teichoic acid, vitamins, amino acids, and antimicrobial peptides, lipopolysaccharides, exopolysaccharides, enzymes, bacterial lysates (a mixture made from bacterial components), and cell-free supernatants (a mixture of compounds produced by bacteria and yeast). A skilled artisan can determine the appropriate amount of food ingredients, vitamins, minerals, probiotics, prebiotics, postbiotics, antioxidants, or other ingredients to be used to make a particular composition to be administered to a particular animal. Such artisan can consider the animal's species, age, size, weight, health, and the like in determining how best to formulate a particular composition comprising such ingredients. Other factors that may be considered include the desired dosage of each component, the average consumption of specific types of compositions by different animals (e.g., based on species, body weight, activity/energy demands, and the like), and the manufacturing requirements for the composition.

In a further aspect, the present disclosure provides kits suitable for administering food compositions to animals. The kits comprise in separate containers in a single package or in separate containers in a virtual package, as appropriate for the kit component, one or more of (1) one or more ingredients suitable for consumption by an animal; (2) instructions for how to combine the ingredients and other kit components to produce a composition useful for providing a health benefit as described herein; (3) instructions for how to use the food composition to obtain such benefits; (4) one or more probiotics; (5) one or more inactivated probiotics; (6) one or more components of inactivated probiotics that promote health benefits similar to or the same as the probiotics, e.g., proteins, lipids, glycoproteins, and the like; (7) one or more prebiotics; (8) a device for preparing or combining the kit components to produce a composition suitable for administration to an animal; and (9) a device for administering the combined or prepared kit components to an animal. In one embodiment, the kit comprises one or more ingredients suitable for consumption by an animal. In another embodiment, the kit comprises instructions for how to combine the ingredients to produce a composition useful for obtaining a health benefit as described herein.

When the kit comprises a virtual package, the kit is limited to instructions in a virtual environment in combination with one or more physical kit components. The kit contains components in amounts sufficient to obtain a health benefit as described herein. Typically, the kit components can be admixed just prior to consumption by an animal. The kits may contain the kit components in any of various combinations and/or mixtures. In one embodiment, the kit contains a container of food for consumption by an animal. The kit may contain additional items such as a device for mixing ingredients or a device for containing the admixture, e.g., a food bowl. In another embodiment, the food compositions can be mixed with additional nutritional supplements such as vitamins and minerals that promote good health in an animal. The components can be each provided in separate containers in a single package or in mixtures of various components in different packages. In some embodiments, the kits comprise one or more other ingredients suitable for consumption by an animal. In one aspect, such kits can comprise instructions describing how to combine the ingredients to form a food composition for consumption by the animal, generally by mixing the ingredients or by applying optional additives to the other ingredients, e.g., by sprinkling nutritional supplements on a food composition.

In a further aspect, a means for communicating information about or instructions for one or more of (1) using a food composition for obtaining one of the health benefits described herein; (2) contact information for consumers to use if they have a question regarding the methods and compositions described herein; and (3) nutritional information about the food composition can be provided. The communication means can be useful for instructing on the benefits of using the present methods or compositions and communicating the approved methods for administering food compositions to an animal. The means comprises one or more of a physical or electronic document, digital storage media, optical storage media, audio presentation, audiovisual display, or visual display containing the information or instructions. In one aspect, the means can be selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, methods for manufacturing a food composition comprising one or more other ingredients suitable for consumption by an animal, e.g., one or more of protein, fat, carbohydrate, fiber, vitamins, minerals, probiotics, prebiotics, and the like, can comprise admixing one or more of the ingredients suitable for consumption by an animal. The composition can be made according to any method suitable in the art.

In another aspect, a package useful for containing compositions described herein can comprise at least one material suitable for containing the food composition and a label affixed to the package containing a word or words, picture, design, acronym, slogan, phrase, or other device, or combination thereof that indicates that the contents of the package contains the food composition. In some embodiments, the label affixed to the package contains a word or words, picture, design, acronym, slogan, phrase, or other device, or combination thereof that indicates that the contents of the package contain the food composition with beneficial properties relating to a health benefit described herein. In one aspect, such device can comprise the words “improves skin and coat,” enhances eye health,” or an equivalent or similar expression printed on the package. Any package configuration and packaging material suitable for containing the composition can be used herein, e.g., bag, box, bottle, can, pouch, and the like manufactured from paper, plastic, foil, metal, and the like. In one embodiment, the package contains a food composition adapted for a particular animal such as a human, canine, or feline, as appropriate for the label, in one aspect, a companion animal food composition for dogs or cats. In one embodiment, the package can be a can or pouch comprising a food composition described herein. In various embodiments, the package further comprises at least one window that permits the package contents to be viewed without opening the package. In some embodiments, the window can be a transparent portion of the packaging material. In others, the window can be a missing portion of the packaging material.

### EXAMPLES

The invention can be further illustrated by the following example, although it will be understood that this example is included merely for purposes of illustration and is not intended to limit the scope of the invention unless otherwise specifically indicated.

### Example 1—Canine Study

Thirty dogs with a body condition score (BCS) of 7 or higher were recruited in this weight loss study. Each dog's baseline maintenance energy requirement (MER) was determined as the amount of food that stabilized the dog's body weight (less than 5% change over three weeks). Then, each dog's baseline % body fat was determined by DEXA. The dogs were randomized into 2 groups based on baseline body weight, MER, age, gender and % body fat with 15 dogs per group.

Dogs in both groups were fed 75% of their baseline MERs during the first 4 months of the study and 60% of their baseline MERs during the last 2 months of the study. Each group of dogs were fed either the test diet or control diet as shown in Table 1.

TABLE 1

| Components | Test Diet | Control Diet |
|---|---|---|
| Moisture (% as fed) | 8.07 | 8.09 |
| Protein (% as fed) | 48.70 | 26.47 |
| CHO (% as fed) | 22.19 | 34.12 |
| Starch (% as fed) | 15.65 | 31.60 |
| Fat (% as fed) | 10.1 | 14.73 |
| Crude fiber (% as fed) | 5.0 | 11.4 |
| ME (kcal/kg) | 3150 | 3181 |

As shown in Table 1, the test diet was high protein, low carb diet with protein to starch ratio of 3:1, while the control diet was a typical weight loss diet having moderate protein and moderate carbohydrates with protein to starch ratio of 0.84:1. Notably, the two diets had comparable metabolizable energy (ME), but differed in protein, starch, carbohydrates, fat and fiber.

Serum samples were collected at baseline, 2, 4 and 6 months of the study. Blood samples were used to measure blood protein profiles in the dogs. Specifically, blood type collagen data is shown in Table 2.

TABLE 2

| Collagen Type | Increase | No Change |
|---|---|---|
| Collagen Type I alpha-1(I) chain: | X | — |
| C-term propeptide | | |
| Collagen Type III | X | — |
| Collagen alpha-1(V) chain | X | — |
| Collagen alpha-3(VI) chain | — | X |
| Collagen alpha-1(XXVIII) chain | — | X |
| Collagen alpha-1(VI) chain | — | X |
| Collagen alpha-5(VI) chain | — | X |
| Collagen alpha-3(VI) chain | — | X |
| Collagen alpha-1(IX) chain | — | X |
| Collagen alpha-1(X) chain | — | X |
| Collagen alpha-2(VI) chain | — | X |
| Collagen alpha-1(VI) chain | — | X |
| Collagen alpha-3(IX) chain | — | X |
| Collagen alpha-1(XXIII) chain | — | X |
| Collagen alpha-1(VIII) chain | — | X |
| Collagen alpha-1(XIII) chain | — | X |
| Collagen alpha-1(IX) chain | — | X |
| Collagen alpha-1(XXV) chain | — | X |
| Collagen alpha-1(XV) chain | — | X |
| Collagen alpha-1(XX) chain | — | X |

The results showed that out of many collagens detected in the blood the test diet unexpectedly increased blood levels of type I, III, and V collagen compared with the control diet at the end of the study (month 6). Notably, collagen type I, III, and V are known to be essential for bone, skin, connective tissue, muscle, organs, cornea, and hair.

Notably, the present results were completely unexpected as there were no significant differences in hundreds of other blood proteins, especially 16 other types of collagens analyzed as show in Table 2 as well as hundreds of other compounds such as Interleukin-23, Interleukin-12, Interleukin-31, Interleukin-18 receptor accessory protein, Interleukin-27 subunit beta, Interleukin-4, Interleukin-12 subunit beta, Interleukin-22, Interleukin-17F, Interleukin-13, Interleukin-36 beta, Interleukin-1 family member 10, Interleukin-15, Interleukin-35, Interleukin-6, Interleukin-37, Interleukin-10, Interleukin-16, Interleukin-27, Interleukin-19, Interleukin-1 beta, Interleukin-2, Interleukin-24, Interleukin-8, Interleukin-17A, Interleukin-5, Interleukin-17D, Interleukin-25, Interleukin-20, Interleukin-7, Interleukin-11, Interleukin-34, Interleukin-6, Interleukin-3, Interleukin-1 alpha, Interleukin-18, Interleukin-9, Interleukin-21, Interleukin-36 gamma, Interleukin-17A, and Interleukin-17C.

In the specification, there have been disclosed certain embodiments of the invention. Although specific terms are employed, they are used in a generic and descriptive sense only and not for purposes of limitation. The scope of the invention is set forth in the claims. Obviously, many modifications and variations of the invention are possible in light of the above teachings. It is therefore to be understood that within the scope of the appended claims, the invention may be practiced otherwise than as specifically described.

What is claimed is:

1. A method for increasing type I, III, and V collagen in an animal comprising administering a food composition to the animal, wherein the food composition comprises:
from about 30% to about 65% protein,
from about 0.1% to about 30% starch, and
from about 10% to about 40% fat;
wherein the food composition has a protein to starch ratio of at least 2:1.

2. The method of claim 1, wherein the protein comprises about 40% to 55% of the food composition.

3. The method of claim 1, wherein the starch comprises about 10% to about 20% of the food composition.

4. The method of claim 1, wherein the fat comprises about 10% to about 30% of the food composition.

5. The method of claim 1, wherein the food composition is administered to the animal on a regular basis.

6. The method of claim 1, wherein the ratio of protein to starch ranges from about 2.5:1 to about 4:1.

7. The method of claim 1, wherein the food composition is a pet food composition.

8. The method of claim 7, wherein the pet food composition is complete and balanced.

9. The method of claim 1, wherein the pet food composition is a supplement.

10. The method of claim 1, wherein the food composition is a non-ketogenic food composition.

11. The method of claim 1, wherein the animal is a companion animal.

12. The method of claim 1, wherein the animal is a canine.

13. The method of claim 1, wherein the animal is an aging animal.

14. The method of claim 1, wherein the animal is a senior animal.

15. The method of claim 1, wherein increasing type I, III, and V collagen provides a health benefit to the animal selected from the group consisting of improved skin, improved coat, improved hair, supporting skin health and function, maintaining skin health and function, treating signs of skin aging, reducing or delaying signs of skin aging, treating dermatosis, decrease of itchy skin, improving eye health, supporting or maintaining eye health, improving cornea health, supporting or maintaining cornea health, treating or preventing cornea disease, and combinations thereof.
